(19) United States
(12) Patent Application Publication
(10) Pub. No.: US 2022/0176152 A1
(43) Pub. Date: Jun. 9, 2022

(54) METHOD AND SYSTEM FOR IMPROVING LIGHT INTAKE, LIGHT EXPOSURE, AND LIFESTYLE MANAGEMENT OF A USER

(71) Applicant: Ocutune ApS, Charlottenlund (DK)

(72) Inventors: Joachim Stormly HANSEN, Valby (DK); Daniel BACHMANN, Charlottenlund (DK); Lars FREDERIKSEN, Gentofte (DK); Anne Marie WORNING, Charlottenlund (DK)

(21) Appl. No.: 17/598,931

(22) PCT Filed: Apr. 1, 2020
(86) PCT No.: PCT/DK2020/050084

§ 371 Date: Sep. 28, 2021

(30) Foreign Application Priority Data
Apr. 1, 2019 (DK) PA2019 70204

(51) Int. Cl. A61N 5/06; G16H 10/60

(52) U.S. Cl. CPC A61N 5/0622; G16H 10/60; A61N 2005/0663; A61N 2005/0628; A61N 2005/0657

(57) ABSTRACT

The present invention relates to a system for improving light intake, light exposure, and lifestyle management of a user, which system comprises a measuring device, a control unit, and a receiving device. The invention also related to a method for improving light intake and light exposure of a user.

### FIELD

The present invention relates to the field of light intake of persons, and more specifically to a method and a system for improving light intake, light exposure, and lifestyle management of a user.

### BACKGROUND

The development and spread of artificial light such as electric lighting has led to a development that has revolutionized the modern society. This development has enabled human beings to break the connection to the natural surroundings around them. This 24-hour lighting allows to perform any activities at any time of the day independently from the natural light and dark cycle around, by extending the days artificially, as noted by Lockley. S W.; Foster, Russell G., ‘Sleep A Very Short Introduction’ Oxford University Press 2012. ISBN 978-0-19-958785-8. Many employments can be performed at night and studies show that human beings spend 90% of the time indoor (Leech, J. A., W. C. Nelson, R. T. Burnett, S. Aaron and M. E. Raizenne. 2002. “It's about Time: A Comparison of Canadian and American Time-Activity Patterns.” Journal of Exposure Analysis and Environmental Epidemiology 12(6): 427-32.). Human beings today are therefore more and more becoming an indoor generation. This is in contrast to human history, where a more outdoor lifestyle was taking place and therefore a completely different light exposition pattern. The light exposition patterns are evolving faster than the circadian rhythm can adapt to this new situation. The consequences of this modern lifestyle are that human beings are less exposed to daylight, are becoming less synchronized with their circadian rhythm, getting less sleep or sleeping at wrong or shifted times, often resulting in a social jetlag between human beings as discussed by Marc Wittmann, Jenny Dinich, Martha Merrow, and Till Roenneberg SOCIAL JETLAG: MISALIGNMENT OF BIOLOGICAL AND SOCIAL TIME, Chronobiology International, 23(1&2): 497-509, (2006). The circadian rhythms of human beings are becoming shifted from their surrounding's natural light pattern. This results in that many human beings are awake and sleeping at shifted times compared to the natural light rhythm. Furthermore, human behaviour in general may suffer from this, and result in performing activities such as eating, exercising, or socialising at odd times of the day as discussed by Till Roenneberg, Karla V. Allebrandt, Martha Merrow, and Céline Vetter Social Jetlag and Obesity, Current Biology 22, 939-943, May 22, 2012. This can have an impact on health, well-being, and awareness. With a reduced sleep and sleep quality, the hormone levels in the human body may be disturbed and also affect concentration ability and memory consolidation. It is therefore a challenge for human beings spending most of their time indoor to achieve a sufficient light intake in order to avoid suffering from the above consequences.

Light intake and especially the influence of blue light from for example computer screens or smartphones on humans and their sleep is a well-known problem. Furthermore, it is also known to measure light intake such as blue light emitted from monitors, screens, and further artificial light (Lockley S W, Brainard G C, and Czeisler C A (2003) High sensitivity of the human circadian melatonin rhythm to resetting by short wavelength light, Journal of Clinical Endocrinology and Metabolism 88(9): 4502-4505.). Such a known device is disclosed in US 2018/264224.

A challenge of known systems is that it is not possible to monitor the comprehensive light intake and light exposure of users.

### SUMMARY OF THE INVENTION

On this background, an object of the present invention is to improve the light intake of a user.

According to a first aspect of the present invention, these and further objects are achieved by a method for improving light intake and light exposure of a user, the method comprising the steps of: obtaining light intake data via a measuring device with a control unit, said measuring device being configured to measure light intake over time; determining a light intake goal of the user based on one or more parameters obtained by the control unit; processing said obtained data with the control unit, which processing comprises the steps of: determining a light intake level by comparing the light intake data measured by the measuring device with light intake response curves from one or more human photo pigments and/or photo receptors; converting said light intake level with a converting factor; comparing the converted light intake level to the light intake goal to determine whether the user has reached said light intake goal; determining an instruction with the control unit based on one or more of the previous steps and/or the one or more parameters; and transmitting the instruction to a receiving device with the control unit based on one or more of the previous steps and/or the one or more parameters.

In the method of the invention the control unit transmits an instruction. In the context of the invention, “transmission” may also be referred to as “sending”, and the two terms may be used interchangeably.

By having a method for improving light intake and light exposure of a user according to the invention it is possible to predict, monitor, and improve the light intake and light exposure of a user. On top of that, a more precise unfold of the circadian complexity and a greater discrimination in personalized data is provided. Individual specific advice may be provided to the users, e.g. to achieve individual health and well-being, help with elite sports, help with night shift work, navigating UV-index levels for skin protection, or improving productivity and mood. The invention may also be used on several users, such as a group of users. This may e.g. be in an office, elderly homes, schools, or hotels, health care. This may allow to improve outdoor lighting pollution in neighbourhoods, timing of medicine, jet-lag planning and adaption to mitigate jet-lag or improve sleep hygiene for better learning for a group of people.

The measuring device may be arranged to measure the light intake over any portion of the electromagnetic spectrum, e.g. visible light such as a wavelength in the range of 380 nm to 720 nm. There may be one or more measuring devices. The measuring device may be operatively coupled to the control unit, the receiving device, and/or other measuring devices if there is more than one. The measuring device may e.g. comprise a spectrometer, spectrophotometer, and/or a photodetector of any kind. The measuring device may be able to measure a cumulative light intake over a time interval but may alternatively also be able to measure the light intake continuously over time. The importance of this has been described by Marc Hébert, Stacia K. Martin, Clara Lee, and Charmane I. Eastman, The effects of prior light history on the suppression of melatonin by light in humans, J Pineal Res. 2002 November; 33(4): 198-203, which is incorporated by reference herein.

The measuring device may e.g. be a wearable device that a user is wearing while the measurement is ongoing. Alternatively, the measuring device may be installed or mounted at a fixed position, for example in a room where the user is positioned, preferably such that the measuring device is substantially exposed to the same light as said user. If a plurality of measuring devices is present e.g. a measuring device as a wearable device on the user or a mobile phone and a measuring device installed in the room where the user is present in e.g. an office or a living room, the data from the different measuring devices may be obtained and processed, such that the light intake of the user may be estimated in the most accurate way. The measuring device may be any type of wearable device such as a ring, a watch, a patch, a necklace, a cap, clothes with an embedded wearable device, or any kind of accessory. The measuring devices for measuring light intake that are to be worn by a user, should preferably be worn such that they are substantially not covered by any clothes or any other shadowing object, in order to get substantially the same light exposition as the user.

The measuring device may also be comprised in a wireless transmit receiver unit (WTRU) such as a mobile phone, tablet, or smartwatch. The measuring device may e.g. comprise or use elements of a WTRU such as a camera which may be used as spectrometer or photodetector or be comprised in a WTRU, such that the measuring device is comprised or is part of e.g. a mobile phone or a smartwatch. The measuring device may be connected wirelessly or electrically to the control unit. The measuring device may comprise a storing device for storing the measured light intake data. This may for example be if the measuring device is not connected to the control unit while measuring light intake, the control unit may thereby obtain the measured data when the measuring device is connected to the control unit again. The measuring device is preferably exposed to light in a similar way that the user is. The measuring device may additionally comprise a GPS. This has the advantage that the location of the user may be tracked e.g. when the user moves from one location to another such as from home to work, travels to another city or country, or goes inside or outside. The location of the user may have an influence on the light exposure of the user and thereby also his light intake. As noted by Roenneberg, T., Kumar, C. J., and Merrow, M. (2007), The human circadian clock entrains to sun time, Curr. Biol. 17, R44-45, which is incorporated by reference herein.

The control unit may thereby obtain location information for the user at any time, and in response to that, determine the light intake goal more precisely and determine the instruction to be transmitted to the receiving device in a more precise and personalized way. The control unit may transmit an instruction to a receiving device, such as a lamp in the room where the user is in order to regulate, compensate, or monitor the light intake of the user.

The obtained light intake data for a time interval may comprise light intake data for a natural light source only, for an artificial light source only, or for a combination of light sources, depending e.g. on the time interval that is measured for and/or the activities and exposition of the user. The user may e.g. have spent an entire day or time interval that was measured on, outside under natural light exposition or inside under artificial light. The light intake data may comprise substantially all or part of the measured light intake, such as several properties relating to the light the measuring device was exposed to. This may be light intensity over time, wavelength, lumens, irradiance vs wavelength, photopic sensitivity, melanopic sensitivity, timing of the light, light vs dark ratio etc.

The light intake goal of the user is determined based on one or more parameters. The light intake goal may e.g. be determined as light intensity over time, wavelength, lumens, irradiance vs wavelength, photopic sensitivity, or melanopic sensitivity. The importance of which is described by S W Lockley, Brigham and Women's Hospital and Harvard Medical School, Boston, Mass., USA, Circadian Rhythms: Influence of Light in Humans, Encyclopaedia of Neuroscience (2009), which is incorporated by reference herein.

The light intake goal of a user may depend on several parameters. The parameter may be related to the user itself, to the behaviour or the habits of the user, or to the geographical location of the user. A parameter may further be a light curve derived from one or more photo pigment or photosensitive receptors of an eye. As noted by Robert J. Lucas et al. Measuring and using light in the melanopsin age, Trends in Neurosciences, January 2014, Vol. 37, No. 1, which is incorporated by reference herein.

Another example of a parameter may also be an environmental factor, such as the season of the year or the weather, whereby the light intake goal may be partly or completely seasonal based, such as a summer light intake goal, a winter light intake goal etc. Some parameters such as geographical and environmental parameters may be determined by means of location data from a connected device such as a mobile phone of a user. The notion of circadian variation in Seasonality, as noted by J.-D. Bergiannaki, T. J. Paparrigopoulos and C. N. Stefanis, Seasonal pattern of melatonin excretion in humans: relationship to daylength variation rate and geomagnetic field fluctuations, Experientia 52 (1996), which is incorporated by reference herein.

A parameter may also be related to the user itself, e.g. as the gender, the age, and/or the chronotype of the user. The chronotype of a user may e.g. be based on the dim light melatonin onset (DLMO) as noted by A. Wirz-Justice HOW TO MEASURE CIRCADIAN RHYTHMS IN HUMANS, MEDICOGRAPHIA, VOL 29, No. 1, 2007, which is incorporated by reference herein, a phase response curve (PRC) of the user, which may e.g. be a phase response curve for a specific light source for a single human or a group of humans. An example of this is disclosed in Khalsa, S. B. S.; Jewett, M. E.; Cajochen, C.; Czeisler, C. A.; A phase response curve to single bright light pulses in human subjects, as it is disclosed in J Physiol (2003), 549.3, pp. 945-952, or Revell, V. L. et al., ‘Human phase response curve to intermittent blue light using a commercially available device’ J Physiol 590.19 (2012) pp 4859-4868, which are incorporated by reference herein, and/or a questionnaire that the user has been subjected to in order to determine e.g. the chronotype of said user which for example is disclosed in Home J A, Ostberg O., A self-assessment questionnaire to determine morningness-eveningness in human circadian rhythms, Int J Chronobiol. 1976; 4(2):97-110, which is incorporated by reference herein. This may allow addressing a more complex and individual response to light and behavioural patterns of users. It may for example allow determination of how much a light source shifts the rhythm of a user e.g. compared to his DLMO, and thereby determine the positive or negative compensation in light intake that may be needed for resynchronising the rhythm of the user.

The light intake goal may e.g. be a daily, a weekly, a monthly, ultradian (less than 24 hours), circadian, day/night ratio, infradian, circalunal rhythm based, seasonal, equinox, summer/winter solstice and/or any determined period light intake goal such as minute interval, hourly etc. Alternatively, the light intake goal may be determined in a live or instant fashion, such that the light intake goal may vary depending on a live parameter and may thereby be updated continuously.

Another way of determining the light intake goal of the user may be based on the historical data that has been obtained. For example, a user having a daily or weekly routine may have a light intake goal based on his routine activities.

The one or more parameters used to determine the light intake goal may also relate to different so called zeitgebers that have an influence on the biological rhythm of humans, as noted by T. Roenneberg and M. Merrow, Entrainment of the Human Circadian Clock, Cold Spring Harb Symp Quant Biol 2007 72: 293-299, which is incorporated by reference herein. These zeitgebers may be photic zeitgebers such as the influence of light on the biological rhythm of the user, but may also be non-photic zeitgebers, which are all the external factors that have an influence on the biological rhythm and behaviour of humans. Examples of non-photic zeitgebers, are the sleep-wake cycle, physical activity, meals, social time, medication, temperature etc. The determination of the light intake goal may e.g. be based on a combination of photic zeitgebers and non-photic zeitgebers. As noted by Anna Wirz-Justice and Colin Fournier, What is the impact of chronobiology on design, particularly on architecture? World health design 2010, which is incorporated by reference herein.

Furthermore, by comparing the measuring light intake with light intake response curves from one or more human photo pigment and/or photo receptors it is possible to determine a more precise impact of the light the user was exposed to with regards to the user's circadian rhythm.

The processing of the obtained data from the measuring device comprises the steps of determining a light intake level based on the obtained light intake data, converting said light intake level with a converting factor, and comparing the converted light intake level to the light intake goal, to determine whether the user has reached said light intake goal. The converting factor may be in the range of 0.1 and 5, preferably between 0.2 and 2, more preferably between 0.5 and 1.5, and most preferably between 0.8 and 1.2. The converting factor for converting a melanopic lux (z-lux), S-cone-opic, M-cone-opic, L-cone-opic, and/or Rhodopic, and/or melanopic action factor value (a_{mel,v}) of a given light source to a MDEI D65 or EDI may for example be 0.906 and 1.104 from MDEI D65 to melanopic lux. The converting factor may be determined by the control unit based on the unit of the light intake goal and the light intake level. In some situations, the light intake level and the light intake goal may be compared without substantially converting the light intake level e.g. if the light intake level and the light intake goal substantially have the same unit. As noted by The SSL D3.7 REPORT, 2016, and CIE TN 003:2015—Report on the First International Workshop on Circadian and Neurophysiological Photometry, 2013, and CIE S 026-CIE-S-026-EDI-Toolbox-Userguide, all of which are incorporated by reference herein.

The steps are preferably consecutive but may alternatively also be in another order.

The light intake level may be determined based on one or more properties of the light intake data. The light intake level may comprise one or more levels such as light intake levels for different properties of the light intake data. The light intake level may comprise one or more physical units depending on the light intake level that is determined. In order to be able to determine or estimate the impact of the measured light e.g. on the users the determined light intake level is converted with a converting factor. By converting the light intake level, the light intake level may be normalized such that the light intake level and the light intake goal may be compared, and thereby allow for the determination of whether the user has reached the light intake goal in a more precise and personalized manner. The converting factor may e.g. be different for converting light intake data depending on the nature of the light i.e. natural or artificial, type of outdoor sky e.g. as cloudy, sunny, foggy etc., type of artificial light e.g. as candle light, projector, light emitting diode (LED), computer screen, colour temperature etc.

Based on one or more of the previous steps and/or one or more parameters, the control unit determines the instruction that may be transmitted to the user, depending for example on the result of the comparison of the light intake level and the light intake goal e.g. determining the light intake or the type of light intake that the user has to be subject to if he wants to achieve his light intake goal. A type of instruction may be an advice to the user such as to go outside for a certain period in order to achieve a light intake goal, but may also be an instruction to a receiving device such as a lamp in a room to increase or decrease a light intensity or colour temperature of the lamp e.g. to respectively either boost the light intake of a user e.g. in the morning or reduce the light intake of a user e.g. before going to bed. The light intake and exposure of the user may thereby be improved on an individual level e.g. by compensating for too much or not enough light intake for a user. An instruction may e.g. be based on the weather outside and advise the user to go out at a strategic moment where the sky is clear, in order to get an improved light intake. A further example may be to transmit an instruction to a receiving device such as automatic curtains in a home that may, based on the time at which the user wakes up in the morning, open up gradually e.g. to perform a dawn/dusk simulation, or in another scenario at night in order to diminish the light pollution from streetlight on the user. An instruction may also be configured such as to divide a day of the user into one or more episodes. These episodes may be related to the natural circadian rhythm of humans in general or may be fitted to the specific user depending on the one or more parameters. These episodes may for example be as follows: dawn/dusk simulation, phase shifting, active day, sunset, active afternoon or evening that e.g. should have an impact on the sleep later on, going to bed soon, sleeping, night. These episodes may be divided into more or fewer steps as said depending on the user, the season, weekday or weekend etc. It may thereby be possible to determine at which time of the day the user gets his light intake by comparing the light intake with the different episodes. Also, a day/night ratio of the light intake may be estimated, which may give an indication on the rhythm and the distribution of the light intake of the user. In order to determine whether the light intake goal has been reached for the user, a point system may be used. This point system may e.g. be linked to the episodes, so that depending on the light intake of the user at the different episodes a certain number of points may be given. The instruction transmitted to the receiving device may further be based on light intake thresholds, such as a night intake threshold at night or a sleepiness threshold or wakefulness threshold during the day. This may for example be if the light intake that is measured is above a certain threshold when approaching sleeping time of a user. Values for the night intake threshold may e.g. be in the range of 0 to 10, 0 to 5, 1 to 4, or 2 to 3 MDEI D65 or EDI. The subjective sleepiness and wakefulness thresholds may be based on the karolinska sleepiness scale (KSS) and e.g. have an equivalent in MDEI D65 or EDI. This may e.g. be KSS≥5 if the user does substantially feel sleepy or KSS≤4 if the user does substantially not feel sleepy e.g. feels alert. Based on this, an instruction of increasing or decreasing the light may be advised, e.g. as described using the scale in: Akerstedt, T. and Gillberg, M., Subjective and objective sleepiness in the active individual, International Journal of Neuroscience, 1990, 52: 29-37, which is incorporated by reference herein.

The determination of the instruction may also be done based on non-photic zeitgeber, such as sleep-wake cycle, physical activity, meals, social time, medication, temperature etc. This may e.g. be behavioural instructions such as when the user is advised to eat, to perform physical activity etc. An example of this could be to advise the user to take his last meal not later than 2-4 hours before going to sleep, or to advise the user to eat all his meals within 8-12 hours for one day, e.g. as noted by G. C. Melkani and S. Panda J Physiol, Time-restricted feeding for prevention and treatment of cardiometabolic disorders, J Physiol 595.12 (2017) pp. 3691-3700, which is incorporated by reference herein.

By obtaining the one or more parameters it may be possible to determine the instruction based on a prediction of the control unit. This prediction may e.g. be based on the behavioural patterns of the user, such as when he sleeps, eats, works, travels, does physical activity etc.

When the instruction to be transmitted has been determined, it is transmitted to the receiving device. The control unit may thereby transmit advice, information, and/or control the receiving device based on the instruction. The receiving device may be a medium for interacting with the user e.g. a computer, a smartphone, or a smartwatch that may inform the user about a status of light intake for a determined period or advice to the user relating e.g. to his light intake, such as advice to increase or decrease the intensity of a light source in the room where the user is, or to go outside e.g. if the light intensity outside is estimated to increase the light intake of the user in a faster or better manner. The control unit may get information about the environment of the user, such as the weather outside by being connected to the internet. As exemplified above, the receiving device may also be an environmental controlling device such as a lamp, curtains, or a network of several environmental controlling devices, e.g. being able to control the environment of the user depending on where the user is located. The light intake and light exposure of the user may thereby be improved by compensating for too high or too low light intake or exposure, based e.g. on the light intake goal. This may e.g. be done by simulating the light exposure of a specific outdoor illumination such as a sunny sky, a specific wavelength or colour temperature or any other type of natural light, and thereby compensating for a user that e.g. did not have the possibility to go outside, has a shifted rhythm e.g. due to his job or travels, at winter time, when the sky is cloudy or generally when the light outside is not sufficient to achieve the light intake goal. There may be a plurality of receiving devices communicating with the control unit, and the receiving devices may also communicate with each other. In some embodiments, the receiving device, the measuring device, and/or the control unit may be comprised in one or more devices. The control unit may be able to control a receiving device such as a lamp, curtains, etc. to improve the light intake and light exposure of the user.

The control unit may be operatively connected to the different elements of the invention, such as the measuring device and the receiving device. The control unit may be wirelessly connected to the different elements of the system. Furthermore, the control unit may be connected to an external network, where the network may e.g. be a network within one or more rooms where the user is or a global network such as the internet such that the control unit may operate from anywhere. The control unit may have access to a database comprising e.g. the one or more parameters, historical data about light intake data, light intake goals, light intake levels, converting factors, and/or instructions. The control unit may be able to log the light intake data, light intake goals, light intake levels, converting factors, one or more parameters, and/or instructions to the database.

In some embodiments, the step of processing the obtained data with the control unit further comprises the step of determining whether a light source for the measured light intake data was a natural light source or an artificial light source.

For example, the determination of the nature of the light source may be performed before the determination of the light intake level, e.g. if the light intake level is determined depended on the nature of the light source.

It may be advantageous to determine whether a light source for the measured light intake data was a natural light source or an artificial light source prior to the comparing step in order to characterise and compare the light intake data to the light intake level. This is done in order to evaluate the nature of the light and thereby be able to characterize the light intake data more precisely. The converting factor is preferably based on the determination of the nature of the light source. This may improve the converting factor used to convert the light intake data. A further advantage of this, may be that it is possible to determine the different light sources that the user has been exposed to over time. This allows determining more precisely the impact of a specific light source on the user.

In some embodiments, the converting factor for converting the light intake is based on one or more of the following: melanopic daylight equivalent illuminance (MDEI), melanopic lux (z-lux), α-opic equivalent daylight (D65) illuminance (EDI), and/or a_{mel,v} of a given light source.

By converting the light intake level based on one or more of the MDEI, melanopic lux (z-lux), α-opic equivalent daylight (D65) illuminance (EDI), and/or a_{mel,v} of a given light source it is possible to standardize the determined light intake level and thereby making it more accurate to compare the impact of different light sources on the user. For example, a light source having a value in melanopic lux, S-cone-opic, M-cone-opic, L-cone-opic, and/or Rhodopic may be converted to MDEI or EDI with a converting factor, or the other way around if the light source has a value in MDEI or EDI. The α-opic equivalent daylight (D65) illuminance (EDI) may be determined with the following formulas for the different human photo pigment and/or photo receptors:
α-opic EDI=α-opic irradiance/α-opic ELR for daylight (D65), and or
α-opic EDI=illuminance/α-opic DER,

Where α-opic irradiance is given in e.g. W·m⁻², α-opic ELR is the efficacy of luminous in e.g. mW·lm⁻¹, and α-opic DER is the daylight (D65) illuminance in lux. The formulas may be derived from the user guide to the Equivalent Daylight (D65) Illuminance Toolbox e.g. from The international standard CIE S 026/E:2018, System for Metrology of Optical Radiation for ipRGC-Influenced Responses to Light, CIE, which is incorporated by reference herein.

This conversion may be from melanopic lux to e.g. MDEI D65, which is a standard daylight illumination, or the other way around. Background about quantification of light exposure is disclosed in SSL-erate 2016 D3.7 Report on metric to quantify biological light exposure doses (din:spec 5031-100:2014), which is incorporated by reference herein.

In some embodiments, at least one of the one or more human photo pigment and/or photo receptors is a retinal ganglion cell (RGC), S-cone-opic (sc), α pigment receptor, M-cone-opic (mc), L-cone-opic (lc), rhodopic rods (rh), melanopic Intrinsically photosensitive retinal ganglion cells (ipRGC), a photosensitive retinal ganglion cells (pRGC), and/or a melanopsin-containing retinal ganglion cells (mRGC) receptor.

In some embodiments, at least one of the one or more human photo pigment and/or photo receptors is an ipRGC receptor of the type M1, M2, M3, M4, or M5.

Background about human photo pigment and photo receptors is disclosed in Lucas et. al 2014 Measuring light in the melanopic age and CIE TN003:2015. Report on the First International Workshop on Circadian and Neurophysiological Photometry, 2013, and Lucas et al. 2014/CIE-TN 003:2015/CIE S 026/E:2018 CIE System for Metrology of Optical Radiation for ipRGC-Influenced Responses to Light, which are incorporated by reference herein.

Empirical observations have shown that circadian and other behavioural and physiological responses can display a very distinct spectral sensitivity to light. In humans and non-human primates this has shown a sensitivity in the short-wavelength portion of the visible spectrum (for example from approximately 447 to 484 nm), divergent from that of visual photopic-lux, having a peak sensitivity around 555 nm.

As a consequence, for the Circadian response and direct non-circadian responses, it has been noted that Photopic lux, is inadequate for quantifying light for the non-visual and circadian system, also in combination with a melanopic response. The light intake level may therefore integrate light through all, substantially all, some, or one of the photoreceptors and subtypes of an eye. Such as, but not exclusively isolated to Short-wavelength (S) cones, Medium-wavelength (M) cones, Long-wavelength (L) L-cones, ipRGCs, and Rods e.g. rhodopic rh (rods), e.g. to assess the full complexity of the circadian system and its responses.

Tables 1 to 3 show photometric measures for each of the five photoreceptive input to circadian and neurophysiological light responses in humans. The connection between the different photo receptors, photo pigments and their peak sensitivity wavelength, and distinct illuminance measures are also shown in Tables 1 to 3.

TABLE 1
Lucas et al. 2014

| | | Human retinal photo pigment complement (all weighted) | | | | | |
|---|---|---|---|---|---|---|---|
| | | | | | | α in | |
| | Symbol | Prefix/Unit | Sensitivity | Iₘₐₓ | | Nα(λ) | Curve |
| 1. | E_{sc} | Cyanopic (sc-lx) | S-cone | 419 | nm | sc | N_{sc}(λ) |
| 2. | E_{z} | Melanopic (z-lx)/(m- | Melanopsin | 480/482 | nm | z/m | N_{z}(λ) |
| | | lux) | | | | | |
| 3. | Eᵣ | Rhodopic (r-lx) | Rod | 496.3 | nm | r | Nᵣ(λ) |
| 4. | E_{mc} | Chloropic (mc-lx) | M cone | 530.8 | nm | mc | N_{mc}(λ) |
| 5. | E_{lc} | Erythropic (lc-lx) | L cone | 558.4 | nm | lc | N_{lc}(λ) |

TABLE 2
CIE TN003: 2015.

| | | The photoreceptors of the human retina, their designation and formulae | | | | |
|---|---|---|---|---|---|---|
| | | for α-opic equivalent illuminance, | | | | |
| | | | α-opic spectral | Quantity (α- | Quantity | |
| | | Photopigment | efficiency, N α | opic equivalent | symbol | |
| | Photoreceptor | (α-opic) | (λ) | illuminance) | (E α) | Unit Symbol |
| 1. | Short- | photopsin | Cyanolabe | cyanopic | E_{sc} | cyanopic |
| | wavelength | (sc) | | equivalent | | equivalent |
| | (S) cone | | | illuminance | | lux (sc-lx) |
| 2. | Medium- | photopsin | Chlorolabe | chloropic | E_{mc} | chloropic |
| | wavelength | (mc) | | equivalent | | equivalent |
| | (M) cone | | | illuminance | | lux (mc-lux) |
| 3. | Long- | photopsin | Erythrolabe | erythropic | E_{lc} | erythropic |
| | wavelength | (lc) | | equivalent | | equivalent |
| | (L) cone | | | illuminance | | lux (lc-lux) |
| 4. | pRGC/(or | melanopsin | Malanopic | melanopic | E_{z} | melanopic |
| | ipRGC) | (z) | | equivalent | | equivalent |
| | | | | illuminance | | lux (z-lux) |
| 5. | Rods | rhodopsin | Rhodopic | rhodopic | Eᵣ | rhodopic |
| | | (r) | | equivalent | | equivalent |
| | | | | illuminance | | lux (r-lux) |

TABLE 3
CIE ILL RESPONCES CIE S 026: 2018

| | | | | α-opic EDI = equivalent |
|---|---|---|---|---|
| | | | | daylight (D65) |
| | Response | Index | Photopigment | Illuminance(lx) |
| 1. | S-cone- | sc | S-cone photopsin | MDEI(D65) −> EDI(D65) |
| | opic | | (cyanolabe) | |
| 2. | Melanopic | mel | Melanopsin | |
| 3. | Rhodpic | rh | Rodopsin | |
| 4. | M-cone- | mc | M-cone photopsin | |
| | opic | | (chloralabe) | |
| 5. | L-cone- | lc | L-cone photopsin | |
| | opic | | (erythrolabe) | |

In some embodiments, the light intake goal is determined based on a prediction by the control unit based on the one or more parameters.

In some embodiments, the measuring device comprises one or more of the following: a spectrometer, a spectrophotometer, and a photodetector.

In some embodiments, the measuring device is a wearable device.

In some embodiments, the measuring device is further configured to measure physical movements.

The measuring device for measuring light intake and the measuring device for measuring physical movements may be separate from each other, such as two separate measuring devices. The measuring device for measuring physical movements may thereby be covered or hidden because it is not necessary that it is exposed to light. Such a measuring device may be a wearable device that may be put in a shoe, or a WTRU such as a smartphone or a smartwatch etc.

In some embodiments, the measuring device further comprises one or more of the following: an accelerometer, a gyroscope, and a gyro sensor.

In some embodiments, at least one of the one or more parameters is selected from the list comprising: a light curve derived from one or more pigment or photosensitive receptors of an eye.

In some embodiments, at least one of the one or more parameters is a non-photopic zeitgeber. By having at least one of the one or more parameters being a non-photopic zeitgeber it is possible to improve a meal timing of a user e.g. by coordinating it with circadian timing. This may be combined e.g. with the DLMO of the user to personalize the meal timing and improve the health and wellbeing of the user. It may further be possible to obtain data about the meal timing of the user. The meal timing may be detected automatically and logged by e.g. the measuring device but may alternatively be logged manually by the user. An instruction about the ideal meal timing of the user and/or ideal food intake period may be determined by the control unit based on one or more of the previous steps and/or to the one or more parameters. The non-photopic zeitgeber may also be a parameter related to physical activity of the user, which may also be detected by the measuring unit. This may provide a more precise and personalized instruction to the user in order to improve the light intake and light exposure of the user, and the synchronization of the user to the circadian rhythm.

In some embodiments, the instruction transmitted in the transmitting step comprises an instruction to the user to increase his light intake.

According to a second aspect, the invention relates to a system for improving light intake and light exposure of a user, where the system comprises a measuring device for measuring light intake over time, a receiving device, and a control unit, where the control unit is configured to obtain light intake data from the measuring device, to determine a light intake goal of the user based on one or more parameters; to process the obtained data, where the processing comprises obtaining light intake data for a time interval from the measuring device, determining a light intake level based on the obtained light intake data by comparing the light intake data measured by the measuring device with light intake response curves from one or more human photo pigments and/or photo receptors, converting said light intake level with a converting factor, and comparing the converted light intake level to the light intake goal to determine whether the user has reached said light intake goal, and where the control unit further is configured to determine an instruction based on one or more of the previous steps and/or the one or more parameters, and to transmit an instruction to the receiving device based on one or more of the previous steps and/or the one or more parameters.

According to a third aspect, the invention relates to a measuring device configured to measure light intake over time, wherein said measuring device is further configured to be used in any method according the first aspect of the invention and in any system according to the second aspect of the invention.

According to a fourth aspect, the invention relates to a receiving device configured to be used in any method according the first aspect of the invention and in any system according to the second aspect of the invention, where said receiving device is configured to receive an instruction from the control unit and to notify the user of the instruction.

According to a fifth aspect, the invention relates to a computer readable medium comprising computer readable code, wherein the computer readable medium is configured to implement any method according to the first aspect.

It should be understood that combinations of the features in the various embodiments and aspects are also contemplated, and that the various features, details and embodiments may be freely combined into other embodiments. In particular, it is contemplated that all definitions, features, details, and embodiments regarding the system, the methods, the receiving device, and the measuring device apply equally to one another.

In device claims enumerating several means, several of these means can be embodied by one and the same item of hardware. The mere fact that certain measures are recited in mutually different dependent claims or described in different embodiments does not indicate that a combination of these measures cannot be used to advantage.

Reference to the figures serves to explain the invention and should not be construed as limiting the features to the specific embodiments as depicted.

### BRIEF DESCRIPTION OF THE DRAWINGS

The above and/or additional objects, features and advantages of the present invention will be further elucidated by the following illustrative and non-limiting detailed description of embodiments of the present invention, with reference to the appended drawings, wherein:

FIG. 1 shows a block diagram of a system for improving light intake and light exposure of a user, according to an embodiment of the invention.

FIG. 2 shows a flow diagram of how retinal irradiance is processed by photo pigments and photoreceptors, the figure is reproduced from Lucas et. al 2014 “Measuring light in the melanopic age”.

FIG. 3 depicts the absorbance as a function of wavelength of different photo receptors.

FIG. 4 depicts the melatonin suppression of different distinct illuminance measures and photopic lux, reproduced from SSL-erate D3.7 Report 2016.

FIG. 5 depicts chronotype as a function of age and gender, the figure is reproduced from Roenneberg et al. 2004—Current Biology.

FIG. 6 depicts distinct illuminance measures and photopic lux transmittance between the outer surface of the eye and the retina at the ages 32 and 70, the figure is reproduced from CIE TN003:2015. Report on the First International Workshop on Circadian and Neurophysiological Photometry, 2013.

FIG. 7 depicts a flow chart of an embodiment of the invention according to a first aspect of the invention.

FIG. 8 depicts a block diagram of an embodiment of a receiving device according to a fourth aspect of the invention.

FIG. 9 depicts a block diagram of an embodiment of a measuring device according to a third aspect of the invention.

FIG. 10 depicts a block diagram of an embodiment of a control unit according of the invention.

### DETAILED DESCRIPTION

In the following description reference is made to the accompanying figures, which show by way of illustration how the invention may be practiced.

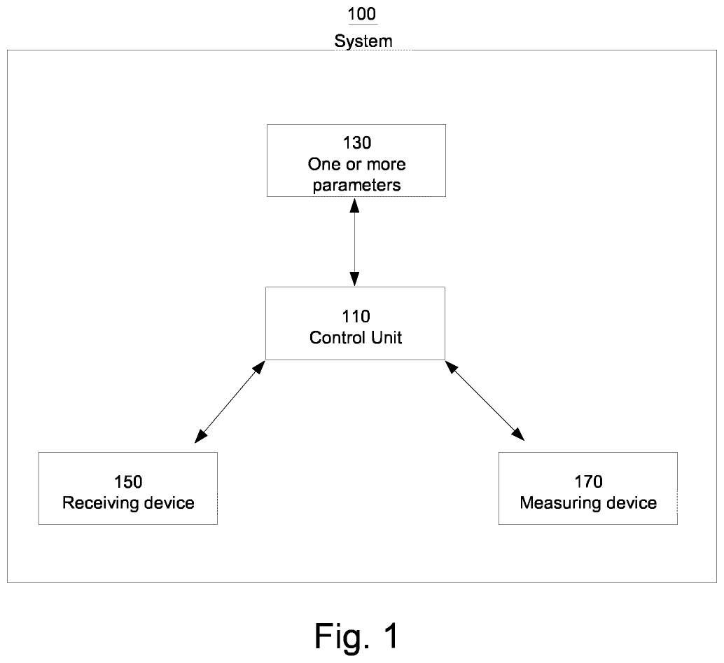

FIG. 1 shows a block diagram of a system 100 for improving light intake and light exposure of a user, according to an embodiment of the invention.

The system 100 comprises a measuring device 170 for measuring light intake over time, a receiving device 150, and a control unit 110, where the control unit 110 is configured to obtain light intake data from the measuring device 170; determine a light intake goal of the user based on one or more parameters 130; process the obtained data, where the processing comprises: to obtain light intake data for a time interval from the measuring device 170, to determine a light intake level based on the obtained light intake data, to convert said light intake level with a converting factor, to compare the converted light intake level to the light intake goal, to determine whether the user has reached said light intake goal, to determine an instruction based on one or more of the previous steps, to transmit an instruction to the receiving device 150 based on one or more of the previous steps. In an embodiment the system 100 is comprised in one device.

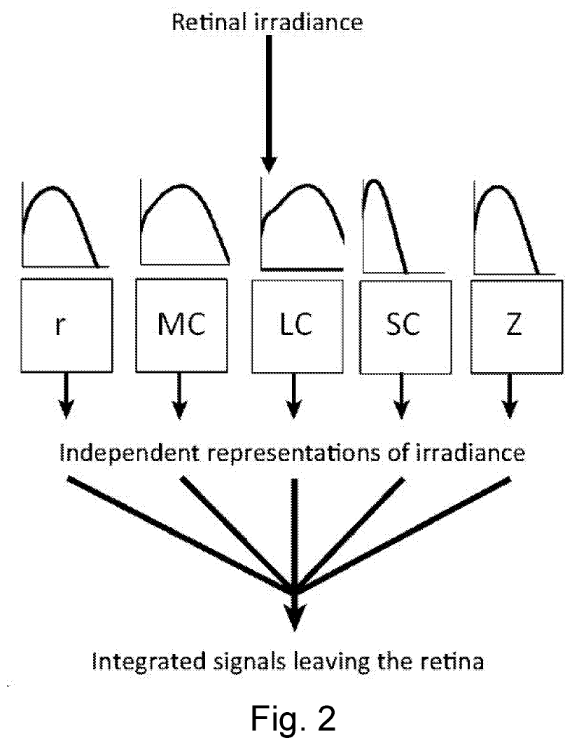

FIG. 2 shows a flow diagram of how retinal irradiance is processed by photoreceptors. Older standards for measuring light intakes for a person have relied on the measurement of photopic lux. Photopic lux describes the average response of the three colour cone photoreceptors. The use of merely photopic lux have proven inadequate and too simple in explaining the body's response to retinal irradiance, especially regarding irradiated lights impact on the circadian system, c.f. Lockley et al. 2003. Therefore, a new and improved model have been developed in explaining how retinal irradiance affects the light in-take for a person. A simple representation of the new model is conceptualized in FIG. 2, where a number of photoreceptive mechanisms are depicted. In FIG. 2 r denotes rod, MC denotes medium-wavelength cone, SC denotes short-wavelength cone, mel(M) denotes pRGC and/or ipRGC, and LC denotes long-wavelength cone, each of which responds to irradiated light according to its own response curve (shown in cartoon form as plots of log sensitivity against wavelength from 400 to 700 nm) to generate a distinct measure of illuminance. Light irradiated onto a retina interacts with photoreceptive mechanisms to create input signals. The created input signals are combined within the ipRGC, to produce a combined signal that is sent to a non-image-forming centres in the brain. This combined signal influences our circadian rhythm and hormone production. The input signals, dependent on their photoreceptive mechanism, are produced by their own unique response curve, the response curve for each photo receptive mechanism defines the input signals, and thereby the wavelength dependence of the combined signal, and hence of downstream responses. Therefore, to estimate the precise impact of retinal irradiance, substantially all, some, or one of the photoreceptors and subtypes thereof of an eye should be considered to assess the full complexity of the impact of irradiated light on the circadian system and its responses.

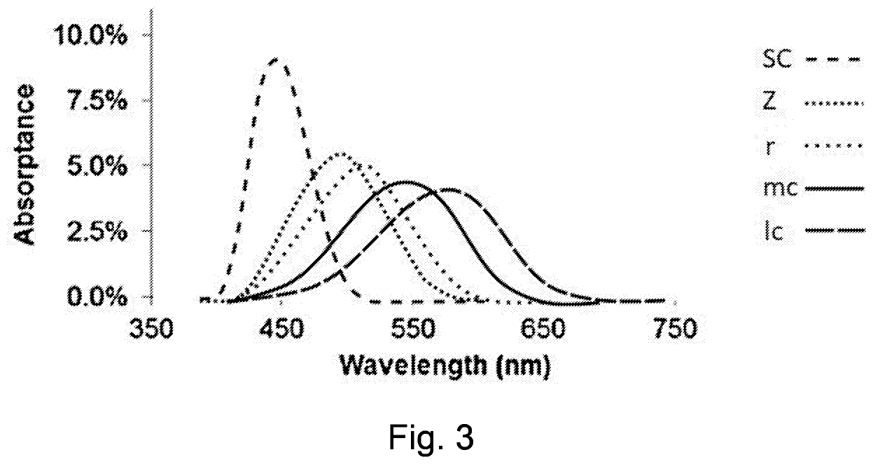

FIG. 3 depicts the absorbance as a function of wavelength of the different photo receptors. From the absorbance it is seen that each photo receptor has a different wavelength, where its peak wavelength sensitivity is located. Even at the respective peak wavelength sensitivities different absorbance are seen for the different photoreceptors. Therefore, in calculating the effective light intake from irradiated light for different photoreceptors/distinct illuminance measures different functions are needed to be used, to account for the unique behaviour of each photoreceptor. Dependent on the context in which the light intake level is calculated, it may only be needed to calculate one or more of the distinct illuminance measures.

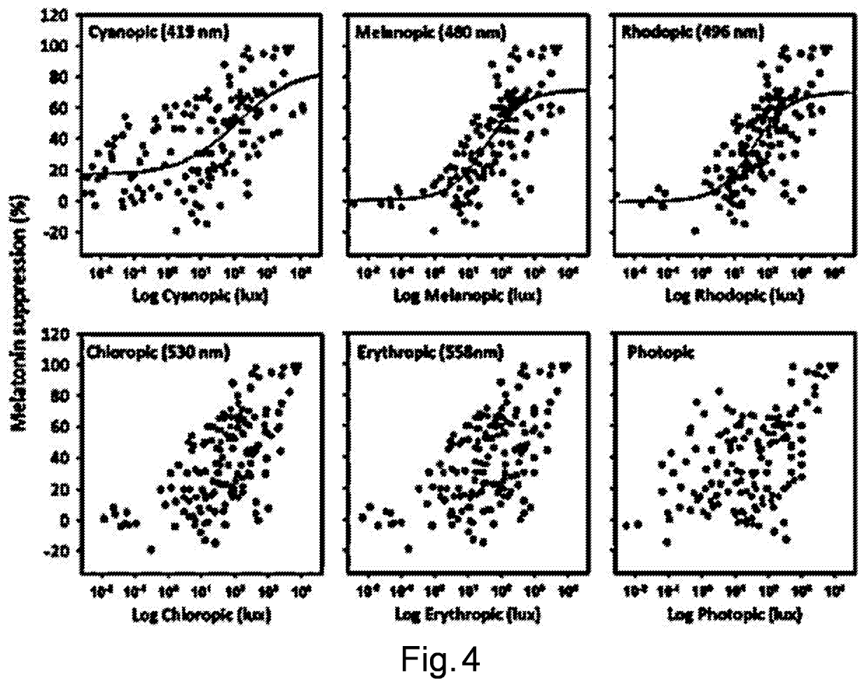

FIG. 4 depicts the melatonin suppression of different distinct illuminance measures and photopic lux. Melatonin is known as a dark hormone and is produced during the night. Melatonin is an important hormone in regulating the circadian rhythm. Melatonin is a physiological signal of night, and as a consequence also a seasonal marker of day-length. What is clearly seen from the depicted graphs on FIG. 4 is that melanopic lux exhibits the strongest correlation with melatonin suppression, whereas photopic lux does not correlate well with melatonin suppression. Therefore, to estimate the impact of irradiated light on the circadian rhythm, measurements of photopic lux may lead to erroneous results for a person's effective light intake and as consequence their light intake goal. Instead of photopic lux, melanopic lux may be preferred to measure irradiated lights impact on the circadian rhythm, as melanopic lux correlates better with melatonin suppression and therefore may be preferable for use in determining light intake and/or for determining a light intake goal. Of course, other distinct illuminance measures may be used in combination with melanopic lux in determining light intake, and in some embodiments melanopic lux is not used in determining light intake.

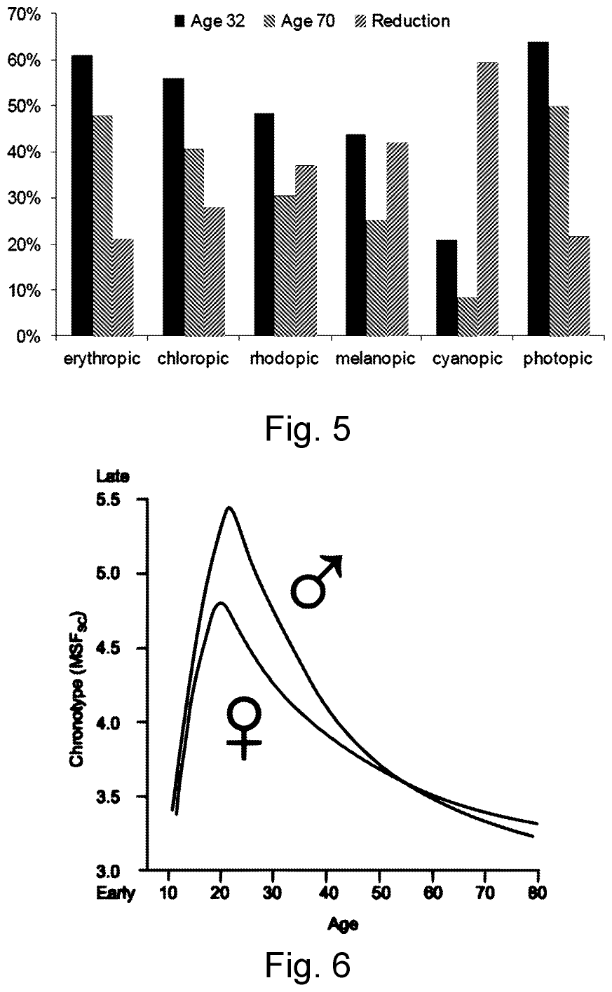

FIG. 5 depicts chronotype as a function of age and gender. The chronotype of a person has been proven to be important in determining the optimal sleep pattern and for the circadian rhythm of the person. The chronotype may vary with parameters, such as age and gender, and even people of the same age and gender may have different chronotypes. Still some trends have been noticed within chronotypes of people. As seen on the graph depicted in FIG. 5 some general trends regarding chronotypes are seen. Children have a generally early chronotype while as they age and become teenagers/young adolescents the chronotype shifts towards a later chronotype, and as the teenagers/young adolescents age and become adults they trend towards an earlier chronotype. As the chronotype of a person affects the circadian rhythm of the person it may be advantageous to include it in determining a light intake goal of the user.

FIG. 6 depicts photopic transmittance between the outer surface of the eye and the retina of different distinct illuminance measures at different ages. As seen in FIG. 6, the transmittance of the distinct illuminance measures changes greatly with the age of a person, with cyanopic lux showing a decrease of nearly 60% between the ages of 32 and 70. Therefore, in determining light intake for a person the age of the person may be highly relevant to include.

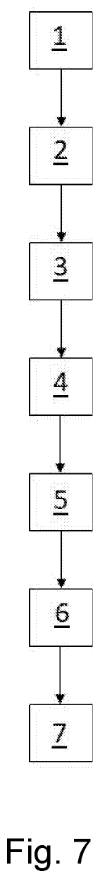

FIG. 7 depicts a flow chart of an embodiment of the invention according to a first aspect of the invention. In the first step 1, light intake data over time is obtained via a measuring device 170. The measuring device 170 may e.g. comprise a spectrometer, spectrophotometer, and/or a photodetector of any kind for obtaining light intake data. The measuring device 170 is configured for transmitting obtained light intake data to a control unit 110. The measuring device 170 may comprise a transmitter or transceiver for transmitting the obtained light intake data. The measuring device 170 may be in the form of a wearable device, e.g. a wristband. The measuring device 170 may also be in the form of one or more stationary sensors. In the second step 2, a light intake goal of a user is determined. The light intake goal is based on one or more parameters 130 obtained by the control unit 110. The one or more parameters 130 may be a non-photopic zeitgeber, a gender, an age and/or a chronotype of the user. The one or more parameters 130 may be obtained via an interface on the control unit 110 allowing the user to manually input the one or more parameters 130. In a third step 3, a light intake level is determined based on the obtained light intake data. Furthermore, determining the light intake level comprises comparing the light intake measured by the measuring device 170 with light intake response curves from one or more human photo pigments and/or photo receptors. By comparing the obtained light intake data with light intake response curves from one or more human photo pigments and/or photo receptors, it is possible to obtain a more accurate light intake level of the user. Since not all incident light is absorbed by photo receptors/photo pigments in the eye. Therefore, not all obtained light intake data contributes to the determined light intake level. In a fourth step 4, the determined light intake level is converted with a converting factor. Converting said light intake level with a converting factor. The converting factor for converting a melanopic lux (z-lux), S-cone-opic, M-cone-opic, L-cone-opic, and/or Rhodopic, and/or melanopic action factor value to a MDEI D65 or EDI may for example be 0.906 and 1.104 from MDEI D65 to melanopic lux. In some situations, the light intake level and the light intake goal may be compared without substantially converting the light intake level e.g. if the light intake level and the light intake goal substantially have the same unit. Conversion of the light intake level is done to more precisely determine or estimate the impact of the measured light on the user. By converting the light intake level, the light intake level may be normalized such that the light intake level and the light intake goal may be compared, and thereby allow for the determination of whether the user has reached the light intake goal in a more precise and personalized manner. The converting factor may e.g. be different for converting light intake data depending on the nature of the light, i.e. natural or artificial, type of outdoor sky, e.g. as cloudy, sunny, foggy etc., type of artificial light e.g. as candle light, projector, LED, computer screen, colour temperature etc. In a fifth step 5, the converted light intake level is compared to the light intake goal, to determine whether the light intake goal has been reached. In a sixth step 6, the control unit 110 determines an instruction based on any of steps one to five 1,2,3,4 and 5. The instruction may be targeted towards the user, e.g. a message telling the user to increase or decrease light exposure. The instruction may be a control of another component communicatively connectable to the control unit 110, e.g. closing or opening of curtains, dimming or brightening of a light emitting device. In a seventh step 7, the determined instruction is transmitted to a receiving device. If the instruction is a message for the user, the receiving device may be a screen for displaying the message, and the screen comprising a receiver for receiving the message.

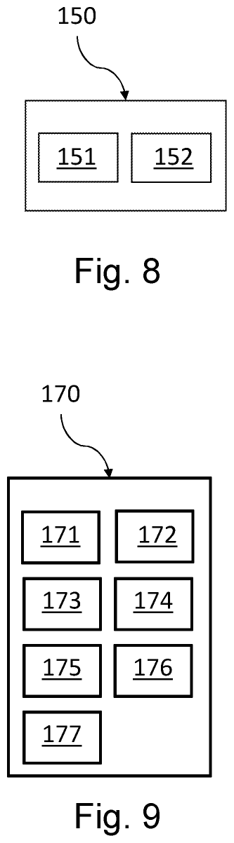

FIG. 8 depicts a block diagram of an embodiment of a receiving device 150 according to the fourth aspect of the invention. The receiving device 150 is configured for receiving the instruction determined by the control unit 110 and notify the user of the received instruction. The receiving device 150 comprises a first receiver unit 151 configured for receiving an instruction determined and transmitted by the control unit 110. The first receiver unit 151 may be a transceiver or a radio receiver. The receiving device 150 further comprises a receiver processing device 152 configured for processing a received instruction. The receiver processing device 152 may further be connected or connectable to an external device, e.g. a display or another processing device. For example, when the instruction is a message for the user, then the message is determined and transmitted by the control unit. The message is received by the receiver unit 151, and subsequently processed by the receiver processing device 152, which may process the message to be shown on the external device.

FIG. 9 depicts a block diagram of an embodiment of a measuring device 170 according to a third aspect of the invention. The measuring device 170 being configured to measure light intake over time. The measuring device 170 comprises an optical sensor 171 configured for measuring incident light. The optical sensor 171 may be a spectrometer, a spectrophotometer, and/or a photodetector. The measuring device 150 further comprises a first transmitter unit 172 configured for transmitting the measured light intake over time. The first transmitter unit 172 is configured for transmitting the measured light intake over time to the control unit 110. The first transmitter unit 172 may be a transceiver or a radio transmitter. The measuring device 170 in the shown embodiment is a wearable device. The measuring device 170 comprises a storing device 173 for storing measured data. Storing of data may be useful if the measuring device 170 is not connected to the control unit 110 while measuring light intake, the control unit 110 may thereby obtain the measured data by reading the storing device 173 when the measuring device 170 is connected to the control unit again. The measuring device 170 comprises a GPS 174, which has the advantage that the location of the user may be tracked e.g. when the user moves from one location to another such as from home to work, travels to another city or country, or goes inside or outside. The measuring device 170 comprises an accelerometer 175. The measuring device 170 comprises a gyroscope 176. The measuring device 170 comprises a gyro sensor 177.

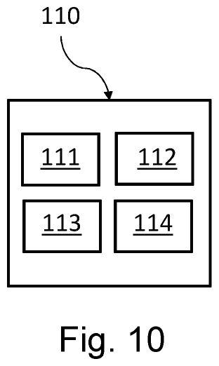

FIG. 10 depicts a block diagram of an embodiment of a control unit 110 according to an embodiment of the invention. The control unit 110 is configured for determining an instruction and transmitting the instruction to the receiving device 150. The control unit 110 comprises a second receiver unit 111 configured for receiving light intake data from the measuring device 170. The second receiver unit 152 may be a transceiver or a radio receiver. The control unit 110 comprises a second transmitter unit 112 configured for transmitting a determined instruction to the receiving device 150. In some embodiments the second receiver unit 111 and the second transmitter unit 112 are comprised within a transceiver unit. The control unit 110 further comprises a controller processing device 113 configured for processing received light intake data. The control unit 110 comprises a database 114. The database may comprise the one or more parameters 130 used in processing the received light intake data. The database 114 may comprise historical data about light intake data, light intake goals, light intake levels, converting factors, and/or instructions. The control unit 110 may log light intake data, light intake goals, light intake levels, converting factors, one or more parameters, and/or instructions to the database 114.

It should be emphasised that the term “comprises/comprising” when used in this specification is taken to specify the presence of stated features, integers, steps or components but does not preclude the presence or addition of one or more other features, integers, steps, components or groups thereof.

1. A method for improving light intake and light exposure of a user, the method comprising the steps of:
obtaining light intake data via a measuring device with a control unit, said measuring device being configured to measure light intake over time;
determining a light intake goal of the user based on one or more parameters obtained by the control unit;
processing said obtained data with the control unit, which processing comprises the steps of:
determining a light intake level by comparing the light intake data measured by the measuring device with light intake response curves from one or more human photo pigments and/or photo receptors,
converting said light intake level with a converting factor,
comparing the converted light intake level to the light intake goal to determine whether the user has reached said light intake goal,
determining an instruction with the control unit based on one or more of the previous steps and/or the one or more parameters; and
transmitting the instruction to a receiving device with the control unit based on one or more of the previous steps and/or the one or more parameters

2. The method according to claim 1, wherein the step of processing the obtained data with the control unit further comprises the step of determining whether a light source for the measured light intake data was a natural light source or an artificial light source.

3. The method according to claim 1, wherein the converting factor for converting the light intake is based on one or more of the following: melanopic daylight equivalent illuminance (MDEI), α-opic equivalent daylight (D65) illuminance (EDI), melanopic lux (z-lux), and/or melanopic action factor (a_{mel,v}).

4. The method according to claim 1, wherein at least one of the one or more human photo pigment and/or photo receptors is a retinal ganglion cell (RGC), S-cone-opic (sc), α pigment receptor, M-cone-opic (mc), L-cone-opic (lc), rhodopic rods (rh), melanopic Intrinsically photosensitive retinal ganglion cells (ipRGC), a photosensitive retinal ganglion cells (pRGC), and/or a melanopsin-containing retinal ganglion cells (mRGC) receptor.

5. The method according to claim 1, wherein at least one of the one or more human photo pigment and/or photo receptors is an ipRGC receptor of the type M1, M2, M3, M4, or M5.

6. The method according to claim 1, wherein the light intake goal is determined based on a prediction by the control unit based on the one or more parameters.

7. The method according to claim 1, wherein the measuring device comprises one or more of the following: a spectrometer, a spectrophotometer, and a photodetector.

8. The method according to claim 1, wherein the measuring device is a wearable device.

9. The method according to claim 1, wherein the measuring device further comprises one or more of the following: an accelerometer, a gyroscope, and a gyro sensor.

10. The method according to claim 1, wherein at least one of the one or more parameters is selected from the list comprising: a light curve derived from one or more pigment or photosensitive receptors of an eye.

11. The method according to claim 1, wherein at least one of the one or more parameters is a non-photopic zeitgeber.

12. The method according to claim 1, wherein at least one of the one or more parameters is a gender, an age, and/or a chronotype of the user.

13. The method according to claim 1, wherein the instruction transmitted in the transmitting step comprises an instruction to the user to increase or decrease his light intake.

14. A system for improving light intake and light exposure of a user, said system comprising a measuring device for measuring light intake over time, a receiving device, and a control unit, wherein the control unit is configured to:
obtain light intake data from the measuring device;
determine a light intake goal of the user based on one or more parameters;
process the obtained data, wherein the processing comprises:
obtaining light intake data for a time interval from the measuring device,
determining a light intake level based on the obtained light intake data by comparing the light intake measured by the measuring device with light intake response curves from one or more human photo pigment and/or photo receptors,
converting said light intake level with a converting factor, and
comparing the converted light intake level to the light intake goal, to determine whether the user has reached said light intake goal,
determine an instruction based on one or more of the previous steps and/or the one or more parameters;
transmit an instruction to the receiving device based on one or more of the previous steps and/or the one or more parameters.

15. (canceled)

16. A receiving device according to claim 14, wherein said receiving device is configured to receive an instruction from the control unit and to notify the user of the instruction.

17. A computer readable medium comprising computer readable code, wherein the computer readable medium is configured to implement the method according to claim 1.
